(19) United States
(12) Patent Application Publication
(10) Pub. No.: US 2014/0052043 A1
(43) Pub. Date: Feb. 20, 2014

(54) Elastic Bandage

(71) Applicant: LOHMANN & RAUSCHER GmbH, SCHONAU AN DER TRIESTING (AT)

(72) Inventors: ERIK STEINLECHNER, Baden (AT); IRIS DISTELRATH, Brohl-Lutzing (DE)

(73) Assignee: LOHMANN & RAUSCHER GmbH, SCHONAU AN DER TRIESTING (AT)

(21) Appl. No.: 13/968,779

(22) Filed: Aug. 16, 2013

(30) Foreign Application Priority Data
Aug. 16, 2012 (EP) 12 005 907.6

(51) Int. Cl. A61F 13/00

(52) U.S. Cl. CPC A61F 13/00029
USPC 602/76; 602/60

(57) ABSTRACT

The invention relates to an elastic bandage for creating a compression dressing comprising foam and a textile material having a textile inner layer that can be placed on the skin and at least one outer layer. The inner layer contains a woven, mesh and/or knit fabric.

### CROSS-REFERENCE TO RELATED APPLICATIONS

This application is based on and claims priority to European Patent Application No. 12 005 907.6, filed on Aug. 16, 2012, which is incorporated herein by reference in its entirety.

### STATEMENT REGARDING FEDERALLY SPONSORED RESEARCH OR DEVELOPMENT

Not applicable.

### BACKGROUND OF THE INVENTION

Conventionally, compression dressings consist of two bandages. An inner bandage (wrap) is placed onto the skin of the patient and is used for skin protection and for cushioning. The bandage can, if necessary, have absorptive properties. A cohesive outer bandage is wrapped around the inner bandage (wrap) and is used for producing a compression effect at a therapeutically prescribed level as well as for maintaining the compression effect over a wearing period of up to seven days. Additionally, the outer bandage is intended for reinforcing the compression dressing and as resistance against any expansion of the underlying layers.

As outer bandages, impregnated textile bandages with elastic threads, such as woven bandages or non-woven bandages, are particularly suitable, because, using them, the required compression level for wrapping the inner bandage is particularly well adjustable.

Bandage or inner bandages, on the other hand, conventionally consist of a foam material that can be placed on the skin, conforms well to the shape of the body part, to which the bandage is applied, and which additionally, due to its cushioning effect, prevents the outer bandage from locally exerting excessive pressure on the patient's body. Moreover, exposed limbs, such as ankles, are protected by the foam layer. In other words, the foam layer in conventional bandages that lies on the skin provides for the pressure exerted by the outer bandage to act uniformly on the bandaged body part.

Document EP 1709947 A1 describes a bandage, the foam layer of which, that can be placed on the skin, is attached to an elastic backing layer that faces away from the skin, said backing layer being impregnated with polymeric binders. As a result of the impregnation, the backing layer acquires cohesive properties, so that the outer bandage that is wrapped around it adheres to it. Hence, such prior art bandages are dual-layer bandages having a skin-facing foam layer and an impregnated backing layer facing away from the skin, onto which, in turn, the outer bandage can be placed. It has, however, been found that the wearing comfort of this bandage substantially decreases after some time. In addition, the foam layer that lies on the skin is perceived as unpleasant by many patients.

For this reason, in prior art compression dressings, a so-called tubular dressing has been arranged between skin and foam layer. In contrast to the bandage that can be wrapped around the limb, the tubular dressing is a tubular sleeve through which the patient can introduce an arm or a leg. It customarily consists of textile materials, which are perceived by the patient as comfortable on the skin. But after applying a compression dressing with tubular dressing, constrictions of the bandaged body part often occur, which are due to the fact that, especially for inexperienced users, whose number is on the increase, this structure of the compression dressing is difficult to handle and, as a result, is highly error-prone in its application.

In other prior art compression dressings, such as those described in document EP 0878179 A2, the foam layer is arranged between a skin-facing absorbent nonwoven layer and the outer bandage. It has, however, been found, that a non-woven layer lying on the skin is prejudicial to lasting wearing comfort and that in many cases the desired effect not sustainably achieved.

### BRIEF SUMMARY OF THE INVENTION

The invention relates to an elastic bandage comprising foam and a textile material to create a compression dressing. The bandage has at least two layers, with a textile inner layer which can be placed on the skin and an outer layer containing foam. Hence, the elastic bandage has a skin-facing textile inner layer to be placed on the skin and an outer layer containing foam.

In view of the described problems, the invention herein is based on the objective of providing an elastic bandage for creating a compression dressing, which improves the wearing comfort, is easy to handle even for inexperienced users, can be applied error-free and can be worn without any problem for an extended period of time and with the desired effect.

This objective is achieved by an enhancement of prior art bandages, which is essentially characterized in that the inner layer of the bandage that can be placed on the skin contains a woven, mesh and/or knit fabric. In other words, the bandage according to the invention has an exposed textile layer to be placed on the patient's skin and is embodied in the form of a woven, mesh and/or knit fabric. This layer differs from a nonwoven fabric particularly in that it consists of fibers spun into yarns or of threads and not of loosely assembled individual fibers.

At least one outer layer, facing away from the skin, of the bandage according to the invention contains a foam material so that the compression pressure the outer bandage to be wrapped around the bandage acts uniformly on the bandaged body part.

The invention is based on the finding that from the prior art bandage of EP 0 878 179 A2 with a non-woven layer that lies on the skin, fibers are released from the non-woven layer, a condition that is further promoted by the development of heat on the skin after extended wearing of the bandage and causes itching. On the other hand, from the bandage according to the invention, with a woven, mesh and/or knit fabric, no fibers are released because these materials are processed in the form of spun yarns or similar, from which individual fibers will hardly be released. With respect to the desired sustained compression effect, the invention is based on the realization that a non-woven fabric very rapidly loses the compression pressure—though slight—that is exerted when the compression dressing is applied, thereby substantially affecting the sustained compression effect of the compression dressing. In contrast thereto, with bandages according to the invention, the slight compression pressure applied initially, at the time of application, is maintained.

It has been found that, as a result of the woven, mesh and/or knit fabric lying on the skin, the wearing comfort is substantially improved and increased, above all when the compression dressing is worn several days. This is particularly due to the fact that the surface of a foam is rough and that a foam layer that lies on the skin causes perspiration due to its nature as a plastic and itching due to the storage of the sweat in the foam. In contrast thereto, woven and/or meshed textile materials are perceived as soft and particularly pleasant on the skin. They hardly cause perspiration or can better absorb and dissipate outward any sweat that forms. They, therefore, have a quasi breathable effect. Hence, the heat accumulation on the skin can be minimized and itching can be reduced as a result of the fluid or vapor dissipation. For this reason, the inner layer of the bandage according to the invention is particularly suitable for direct application to the skin, and even, where appropriate, directly on a relatively small wound, such as, for example on a superficial laceration.

Another advantage of the bandage according to the invention is its ease of handling in applying a compression dressing. In a first step, the body part to be bandaged is wrapped using the bandage, thereby applying the first bandage layer, comprising the inner textile layer and the foam facing away from the skin for cushioning purposes. In a second step, the outer bandage layer is applied for compression purposes. This makes the bandage according to the invention also suitable for inexperienced users and prevents faulty applications of the compression dressing and wrapping errors that can cause dangerous binding or constrictions of the bandaged body part. It was additionally found that, as a result of the woven, mesh and/or knit fabric layer coming to lay on the skin, in association with the outer layer that comprises foam, an improved support function can be achieved.

The bandage according to the invention is not only particularly well suited for compression therapy in phlebology but also in lymphology.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

In the following description, the invention is exemplified with reference to the drawings. In the drawings:

FIG. 1 shows a schematic representation of an elastic bandage according to the invention in its application in the creation of a compression dressing on a body part, and

FIG. 2 shows an exploded view of a two-layer compression dressing according to an additional aspect of the invention.

### DETAILED DESCRIPTION OF PREFERRED EMBODIMENT

In the specification herein, a bandage shall be understood to be a web of material that can be coiled to form a wrap and has a certain length so that, for applying a dressing, it can be wrapped around a body part of a patient. As a result, the dressing is flexible and adaptable to any body contour.

Preferred dimensions of such a material web are, in the direction of the width of the web 8 cm to 12 cm, and in the direction of the length of the web 400 cm to 800 cm.

Preferably, the bandage according to the invention is designed in the form of a long and narrow material ribbon coiled into a roll. Preferably, the roll is coiled in such a way that the inner layer of the bandage that can be placed on the skin points radially outwardly from the roll. This facilitates the unwinding of the bandage around the body part of a patient, during which action the inner textile layer of the bandage comes to lie directly on the skin of the patient. Alternatively, the bandage exists in the non-coiled form of a ribbon or strip, and in this case, it has dimensions of preferably approximately 8 cm to 12 cm in width and approximately up to 1 m in length.

At least one outer layer of the bandage is preferably, at least in sections, embodied by a cushioning layer. Herein, the cushioning layer provides for uniform distribution of the pressure originating from the outer bandage and prevents constriction of the body part to which the dressing is applied.

The cushioning layer may have a foam layer. The foam is preferably an open-cell polyurethane foam. Using a foam material, a pressure acting from the outside can be more evenly distributed than via a cushioning layer of a different type, such as a wadding or non-woven layer. Open-cell polyurethane foam can compensate wrapping errors particularly well and thus avoid constrictions. Expediently, the cushioning layer and the textile layer are embodied as approximately congruent planar formations.

For the purpose of uniform distribution of the compression pressure of the outer bandage and for the purpose of a good cushioning effect, it has proven to be expedient for the cushioning layer to have a thickness of approximately 0.5 mm to approximately 5 mm, preferably from approximately 1 mm to approximately 4 mm.

With a woven fabric as inner layer of the bandage, the following parameters have been found to be particularly suitable for placement on the skin: The woven fabric in its unstretched state should be approximately 40 to 200, preferably 50 to 100 warp threads and/or approximately 40 to 200, preferably 70 to 120 weft threads per 10 cm of woven fabric length or width. This thread density is advantageous in the interest of the wearing comfort, so that contact of the skin with the foam is avoided, but at the same time, sufficient woven fabric elasticity exists. Alternatively or additionally, the fabric may be woven from threads, the thread size of which is greater than 4 tex and less than 20 tex, preferably greater than 6 tex and smaller than 10 tex. This thread size is advantageous in the interest of surface smoothness and thus the wearing comfort of the bandage. The fabric may be woven in a linen, satin and/or twill, and alternatively or additionally, at least in sections, be embodied as leno fabric. By the type of weave, the stretchability and the haptic as well as optical properties of the woven fabric can be controlled. Moreover, the weave affects the grammage and, as a result, the wearing comfort.

With a meshed inner bandage layer, the following parameters have been found to be particularly suitable for application to the skin: The mesh or knit fabric in its unstretched state may have approximately 100 to 500, preferably 200 to 400 stitches in the longitudinal direction per 10 cm and/or in the transverse direction. These stitch densities are advantageous in the interest of absorptivity and stretchability of the material. The mesh or knit fabric may be formed of threads, the thread size of which is greater than 0.05 mm and smaller than 0.5 mm, preferably greater than 0.1 mm and smaller than 0.3 mm. These thread sizes are advantageous in the interest of the surface smoothness and, as a result, also the wearing comfort of the bandage. Depending on the material selected, when selecting nylon material, for instance, the number of stitches and/or thread sizes may also be greater. In any case, in order to avoid contact of the skin with the foam, a substantially closed stitch structure is particularly preferred. As a result of their material structure, knit and mesh fabrics are more stretchable compared to woven fabrics so that the elasticity of the threads used, while maintaining a given material stretchability, may be lower in knit and mesh fabrics than in woven fabrics. The bandage is, therefore, by the selection of the material structure for the inner bandage layer, well controllable with regard to haptic, optic and wearing comfort as well as flexible with regard to adaptability to the body part to be provided with the dressing.

For enhanced breathability properties of the inner layer and, along with it, (removal) and transport of sweat away from the skin, it has proven to be particularly expedient if the woven, mesh and/or knit fabric of the bandage according to the invention contains synthetic fibers, natural fibers and/or a mixture of synthetic and natural fibers. The woven, mesh and/or knit fabric contains materials that are well tolerated by the skin and lie on the skin in a particularly pleasant manner, such as cotton, viscose, polyamide and/or the like. The use of bamboo, for instance, is particularly suitable for an antibacterial effect of the bandage.

A high degree of wearing comfort of the bandage according to the invention can be ensured if the woven fabric, mesh and/or knit fabric has a grammage between 5 g/m² and 80 g/m², preferably between 10 g/m² and 60 g/m², in particular between 25 g/m²g and 45 g/m². A material of a higher grammage would have an adverse effect on stiffness and strength or thickness of the material. With a material of a lower grammage, good fluid removal can no longer be assured in the desired manner, thereby unintentionally increasing the patient's perspiration and along with it, the development of itching.

For obtaining a required bandage stretchability, it has proven to be advantageous if the woven, mesh and/or knit fabric contains elastic threads. In this arrangement, the stretchability of the inner layer that can be applied to the skin can be controlled, on the one hand, via the material structure of the textile layer (type of weave, type of stitches, etc.), on the other hand, via the elasticity of the yarns used.

The application of the bandage according to the invention is particularly simple and little error-prone if the inner layer is connected to at least a single outer layer. This connection may be releasable, for example via adhesion points, or alternatively be non-releasable. A releasable connection has the advantage that the outer layer is reusable after exchanging the inner textile layer.

In a particularly preferred embodiment of the invention, the cushioning layer lies directly on the textile inner layer and is connected with it. Then textile inner layer and cushioning layer can be applied to the patient's body part in a single wrapping step, thereby saving time and preventing errors in applying the dressing.

A non-releasable connection between the textile inner layer and the at least one outer layer can, according to the invention, be achieved by means of flame lamination, welding, in particular ultrasonic welding. Non-releasable connection of the two layers during production of the bandage already saves time and costs.

The bandage according to the invention may have a first outer layer in the form of a cushioning layer connected to the inner layer and, arranged outside the cushioning layer, a second outer layer. The second outer layer may be a second textile layer so that the cushioning layer or foam layer is sandwiched between two textile layers. Alternatively or additionally, the second outer layer may have a cohesive or adhesive surface facing away from the skin. For this purpose, the second outer layer may be coated or impregnated, in particular using a polymeric binder, so that an outer bandage for creating a compression effect adheres to the second outer layer. Preferably, the second outer layer is an impregnated textile layer, such as an impregnated woven fabric layer.

According to a second aspect of the invention, a bandage for creating a compression dressing is provided, comprising foam and a textile material, the bandage having a textile inner layer that can be applied to the skin, a textile outer layer facing away from the skin and disposed between the two textile layers, a cushioning layer. In this arrangement, the textile inner layer is connected to the cushioning layer and the cushioning layer to the outer textile layer. The connections may be releasable or non-releasable.

The textile outer layer may develop a compression effect on the two inner layers and on the body part connected to the bandage. The need of a second outer bandage for producing a compression effect can be omitted in this case. In other words, the at least three-layered bandage according to the invention can be used as a “single-layer compression system” (one component compression system). The application of such a single-layer compression system is particularly simple, fast and little error-prone, because the complete compression dressing is applied in a single wrapping step. The textile inner layer of the single-layer compression system may have been previously described characteristics of the inner bandage layer that can be applied to the skin individually or in a combination with each other. For the reasons explained above related to the first aspect of the invention, the textile inner layer for application to the skin preferably contains a woven, mesh or knit fabric. But it may also, like the outer textile layer, contain or be formed of a “woven” or, however, a “non-woven” material, such as a non-woven fabric or the like.

The elastic bandage according to the first or the second aspect of the invention preferably has a stretchability of more than 20% and less than 100%, preferably more than 40% and less than 70%. Using such a bandage, a slight compression pressure can be particularly easily and sustainably achieved, in particular due to the use of woven, mesh and/or knit fabrics. Depending on the type of application, the need for a second outer compression bandage may be omitted.

In a particularly preferred embodiment of the invention, the stretch response of the bandage involves short traction. In other words, the bandage is a short-traction bandage. Using short-traction bandages, a high working pressure and a low resting pressure can be generated,that may be desired in the case of venous disease, for instance. Short-traction bandages are also effectively used for bandaging open legs, for instance.

Costs can be saved in that the elastic bandage according to the invention is reusable several times. For this reason, the bandage is expediently washable, preferably washable up to a temperature of 95° C.

Depending on the type of application or depending on the type of wound and the body part that is bandaged using the bandage, one of the layers may include skin-care, skin-enhancing, skin temperature regulating, antimicrobial, antiseptic and/or odor-inhibiting substances. The textile inner layer of the bandage may, for instance, contain an antimicrobial and/or antiseptic substance, if it is intended for use for bandaging open legs. Alternatively, the textile inner layer of the bandage may contain a skin care and/or odor-inhibiting substance, if the bandage is intended for use in a case of swollen tissue or an edema. Alternatively or additionally, at least one outer layer, such as a cushioning layer and/or a foam layer, may contain such a substance. This has the advantage that the substance can then be delivered to the skin more slowly, and over a longer period of time. Preferably, however, the skin-facing inner layer contains at least one or a plurality of such substances.

The invention also relates to a two-layer compression dressing having an outer cohesive bandage for generating a compression effect and a skin-facing inner bandage in the form of an elastic bandage. The bandage has textile inner layer that can be applied to the skin and an outer layer containing foam, the inner layer containing a woven, mesh and/or knit fabric.

The bandage may have a second outer layer so that the foam-containing layer is disposed between the textile inner layer and the second outer layer.

Furthermore, the invention relates to a multilayer compression dressing having at least one outer bandage for generating a compression effect and a skin-facing inner bandage in the form of an elastic bandage described above.

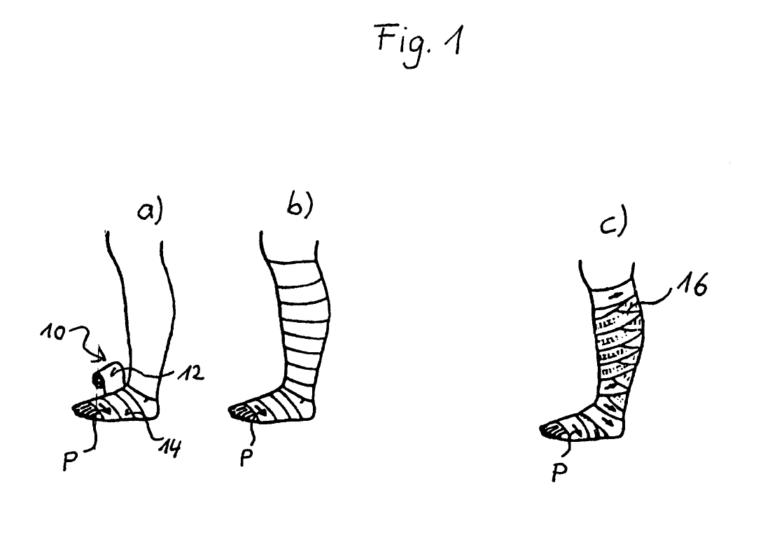

In FIG. 1, the application of an elastic bandage according to the invention for creating a two-layer compression dressing on the lower extremity of a patient is schematically presented. According to FIG. 1a, a bandage 10 in the form of a material ribbon coiled to form a roll is used. The bandage has, facing radially outward in the roll, a textile layer 12 in the form of a woven mesh and/or knit fabric as well as a cushioning layer 14, in the applied state disposed on the outside of textile layer 12, facing away from the patient's skin. The cushioning layer 14 may be embodied congruent with the textile layer 12 and contain an open-cell polyurethane foam. In FIG. 1b, the condition of the patient after applying the bandage is shown. A visual comparison of the FIGS. 1a and 1b makes it obvious that in treating feet and lower legs, it is expedient for the bandage to be wrapped, starting at the toes, first around the foot, then around the ankle joint and then finally around the lower leg. After application of the bandage according to FIG. 1b, the outer bandage, which is used to build up the desired compression pressure, is applied. Outer bandage 16 may also exist in the form of a wrap of a long and narrow material web. It is expediently wrapped in the same wrapping direction as bandage 10, around the patient's lower extremity, as indicated by the arrows P shown in FIGS. 1a and 1c.

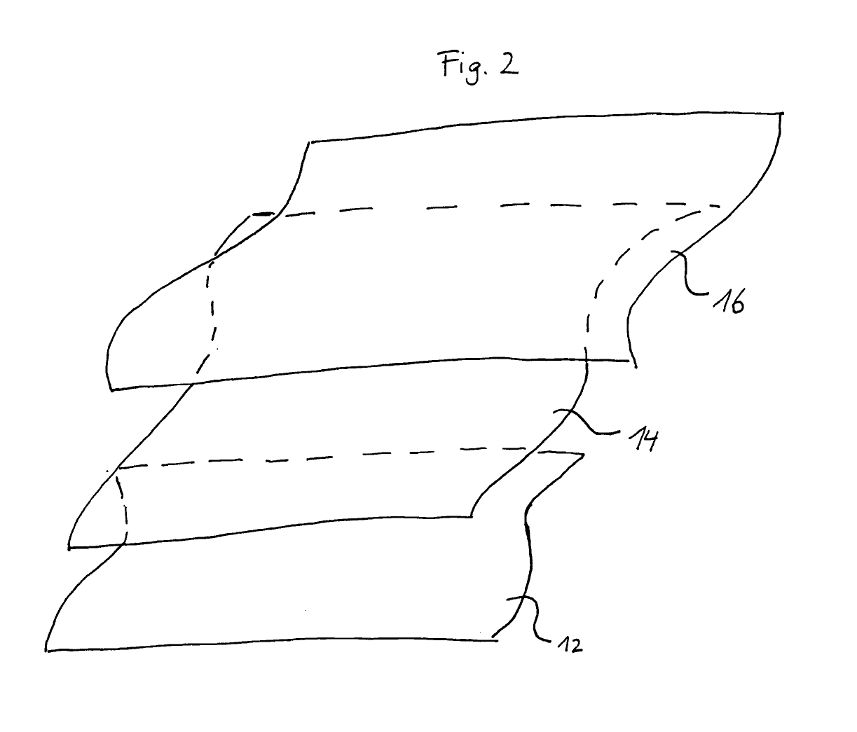

In FIG. 2, a two-layer compression dressing according to the invention is presented. The compression dressing has a textile planar formation 12 that can be placed on the skin, in the form of a woven, mesh and/or knit fabric as well as a cushioning layer 14, and a cohesive bandage 16. The textile layer 12 and the cushioning layer 14 may be connected to each other by adhesion points, by means of ultrasonic welding or else by means of flame lamination. The cohesive bandage is used for applying the desired compression pressure. The bounding surface of the cohesive bandage which faces cushioning layer 14 is expediently embodied in such a way that it adheres well to the bounding surface of cushioning layer 14 that faces away from textile layer 12.

The invention is not limited to the exemplary embodiment explained with reference to the drawings. Within the scope of the invention, it is, for instance, contemplated to coil the textile layer, the cushioning layer and the cohesive bandage into a joint wrap, in order to simplify the application of the bandage in this manner even further. In the exemplary embodiments explained based on the drawings, the cushioning layer consists of an open-cell polyurethane foam. But within the scope of the invention, it may instead consist of two, three or a plurality of components. That may be advantageous for building up a uniform compression pressure. Furthermore, it is contemplated to produce a compression dressing of four, five or a plurality of layers.

1. Elastic bandage for creating a compression dressing comprising foam and textile material having a textile inner layer that can be placed against the skin and at least one outer layer, characterized in that the inner layer has a woven, mesh and/or knit fabric.

2. Bandage according to claim 1, characterized in that, at least in sections, at least one outer layer is embodied by a cushioning layer.

3. Bandage according to claim 2, characterized in that the cushioning layer has a foam layer, preferably comprising an open-cell polyurethane foam.

4. Bandage according to claim 2, characterized in that the cushioning layer has a thickness of approximately 0.5 mm to approximately 5 mm, preferably from approximately 1 mm to approximately 4 mm.

5. Bandage according to claim 1, characterized in that the woven, mesh and/or knit fabric contains synthetic fibers, natural fibers and/or a mixture of synthetic and natural fibers.

6. Bandage according to claim 1, characterized in that the woven, mesh and/or knit fabric has a grammage between 5 g/m2 and 80 g/m2, preferably between 10 g/m2 and 60 g/m2, in particular between 25 g/m2 and 45 g/m2.

7. Bandage according to claim 1, characterized in that the woven, mesh and/or knit fabric contains elastic threads.

8. Bandage according to claim 1, characterized in that the inner layer is releasably connected to at least one outer layer, preferably via adhesive points.

9. Bandage according to claim 1, characterized in that the inner layer is non-releasably connected to at least one outer layer by means of flame bonding, welding, in particular ultrasonic welding.

10. Bandage according to claim 1, characterized by a first outer layer in the form of a cushioning layer connected to the inner layer and a second outer layer arranged outside the first outer layer.

11. Bandage for creating a compression dressing comprising foam and textile material, having a textile inner layer that can be applied to the skin, a textile outer layer facing away from the skin and arranged between both textile layers, a cushioning layer containing foam, characterized in that that the textile inner layer is connected to the cushioning layer and the cushioning layer to the textile outer layer.

12. Bandage according to claim 1, characterized by a stretchability of more than 20% and less than 100%, preferably more than 40% and less than 70%.

13. Bandage according to claim 1, characterized in that the stretching response of the bandage involves short traction.

14. Bandage according to claim 1, characterized by washability up to 95° C.

15. Bandage according to claim 1, characterized in that one of the layers contains skin-care, skin-enhancing, skin temperature regulating, antimicrobial, antiseptic and/or odor-inhibiting substances.

16. Two-layer of compression dressing having an outer cohesive bandage for generating a compression effect and a skin-facing inner bandage in the form of a bandage according to claim 1.

17. Multilayer compression dressing having at least one outer bandage for generating a compression effect, and a skin-facing inner bandage in the form of a bandage according to claim 1.

18. Bandage according to claim 3, characterized in that the cushioning layer has a thickness of approximately 0.5 mm to approximately 5 mm, preferably from approximately 1 mm to approximately 4 mm.
